SEQUENCE LISTING (12 sequences)

SEQ ID NO: 1 - protein, 68 aa, synthetic construct
  REGION 1..68: note = synthetic peptide
EQGAPETLQRCLEENQELRDAIRQSNQILRERCEELLHFQASQREEKEFLMCKFQEARKLVERLGLEK

SEQ ID NO: 2 - protein, 68 aa, synthetic construct
  REGION 1..68: note = synthetic peptide
  VARIANT 7: note = misc_feature - Xaa can be any naturally occurring amino acid preferably a negatively charged amino acid
  VARIANT 12: note = misc_feature - Xaa can be any naturally occurring amino acid, preferably a positively charged amino acid
  VARIANT 32: note = misc_feature - Xaa can be any naturally occurring amino acid, preferably a non-polar or hydrophobic amino acid
  VARIANT 33: note = misc_feature - Xaa can be any naturally occurring amino acid preferably Ala, Ser, or Cys
  VARIANT 35: note = misc_feature - Xaa can be any naturally occurring amino acid, preferably a positively charged amino acid
  VARIANT 52: note = misc_feature - Xaa can be any naturally occurring amino acid, preferably Ala, Ser, or Cys
  VARIANT 63: note = misc_feature - Xaa can be any naturally occurring amino acid, preferably a negatively charged amino acid
  VARIANT 67: note = misc_feature - Xaa can be any naturally occurring amino acid, preferably a non-polar or hydrophobic amino acid
EQGAPEXLQRCXEENQELRDAIRQSNQILREXXEXLLHFQASQREEKEFLMXKFQEARKLVEXLGLXK

SEQ ID NO: 3 - protein, 68 aa, synthetic construct
  REGION 1..68: note = synthetic peptide
EQGAPEELQRCREENQELRDAIRQSNQILREVSERLLHFQASQREEKEFLMAKFQEARKLVEELGLVK

SEQ ID NO: 4 - protein, 62 aa, synthetic construct
  REGION 1..62: note = synthetic peptide
TLQRCLEENQELRDAIRQSNQILRERCEELLHFQASQREEKEFLMCKFQEARKLVERLGLEK

SEQ ID NO: 5 - protein, 62 aa, synthetic construct
  REGION 1..62: note = synthetic peptide
  VARIANT 1: note = misc_feature - Xaa can be any naturally occurring amino acid, preferably a negatively charged amino acid
  VARIANT 6: note = misc_feature - Xaa can be any naturally occurring amino acid, preferably a positively charged amino acid
  VARIANT 26: note = misc_feature - Xaa can be any naturally occurring amino acid, preferably a non-polar or hydrophobic amino acid
  VARIANT 27: note = misc_feature - Xaa can be any naturally occurring amino acid,, preferably Ala, Ser, or Cys
  VARIANT 29: note = misc_feature - Xaa can be any naturally occurring amino acid, preferably a positively charged amino acid
  VARIANT 46: note = misc_feature - Xaa can be any naturally occurring amino acid, preferably Ala, Ser, or Cys
  VARIANT 57: note = misc_feature - Xaa can be any naturally occurring amino acid, preferably a negatively charged amino acid
  VARIANT 61: note = misc_feature - Xaa can be any naturally occurring amino acid, preferably a non-polar or hydrophobic amino acid
XLQRCXEENQELRDAIRQSNQILREXXEXLLHFQASQREEKEFLMXKFQEARKLVEXLGLXK

SEQ ID NO: 6 - protein, 62 aa, synthetic construct
  REGION 1..62: note = synthetic peptide
ELQRCREENQELRDAIRQSNQILREVSERLLHFQASQREEKEFLMAKFQEARKLVEELGLVK

SEQ ID NO: 7 - protein, 73 aa, synthetic construct
  REGION 1..73: note = synthetic peptide
GSWVARLKKELQRCREENQELRDAIRQSNQILREVSERLLHFQASQREEKEFLMAKFQEARKLVEELGLVKLE

SEQ ID NO: 8 - protein, 67 aa, synthetic construct
  REGION 1..67: note = synthetic peptide
GSWELQRCREENQELRDAIRQSNQILREVSERLLHFQASQREEKEFLMAKFQEARKLVEELGLVKLE

SEQ ID NO: 9 - protein, 6 aa, synthetic construct
  REGION 1..6: note = synthetic peptide
VARLKK

SEQ ID NO: 10 - protein, 9 aa, synthetic construct
  REGION 1..9: note = synthetic peptide
GSWVARLKK

SEQ ID NO: 11 - protein, 32 aa, synthetic construct
  REGION 1..32: note = synthetic peptide
GSWSVKELEDKNEELLSEIAHLKNEVARLKKL

SEQ ID NO: 12 - protein, 30 aa, synthetic construct
  REGION 1..30: note = synthetic peptide
ELEDKNEELLSEIAHLKNEVARLKKLVGER

(19) United States
(12) Patent Application Publication
(10) Pub. No.: US 2024/0239851 A1
(43) Pub. Date: Jul. 18, 2024

(54) ENGINEERED NEMO AND USES THEREOF

(71) Applicant: TRUSTEES OF DARTMOUTH COLLEGE, Hanover, NH (US)

(72) Inventors: Maria PELLEGRINI, Hanover, NH (US); Adam H. BARCZEWSKI, Hanover, NH (US); Gevorg GRIGORYAN, Hanover, NH (US)

(21) Appl. No.: 18/514,761

(22) Filed: Nov. 20, 2023

Related U.S. Application Data
(63) Continuation of application No. PCT/US2022/072671, filed on Jun. 1, 2022.
(60) Provisional application No. 63/196,217, filed on Jun. 2, 2021.

(51) Int. Cl. C07K 14/47

(52) U.S. Cl. CPC C07K 14/4703

(57) ABSTRACT

The present disclosure relates to a modified NEMO protein that, inter alia, allows for structural determination of the NEMO protein in the apo-form and in complex with a ligand by NMR or X-ray crystallography. The present disclosure also relates to the use of such modified NEMO proteins to screen and validate inhibitors of the interaction between NEMO and IKKα and/or IKKβ and for structure determination of NEMO in the apo-form and in complex with a ligand, preferably by NMR or X-ray crystallography.

### CROSS REFERENCE TO RELATED APPLICATIONS

This patent application is a continuation of International Application Number PCT/US2022/072671, filed on Jun. 1, 2022, which claims priority to U.S. Provisional Patent Application No. 63/196,217, filed on Jun. 2, 2021, the entire contents of which are fully incorporated herein by reference.

### FEDERALLY SPONSORED RESEARCH OR DEVELOPMENT

This invention was made with government support under R03 AR066130 awarded by the National Institutes of Health and R01 GM133844 awarded by the National Institutes of Health. The government has certain rights in the invention.

### SEQUENCE LISTING

The instant application contains a Sequence Listing which has been submitted in ST.26 XML format via Patent Center and is hereby incorporated by reference in its entirety. Said ST.26 XML copy, created on Apr. 8, 2024, is named “029511-8124 Sequence Listing.txt”, and is 23,790 bytes in size.

### FIELD OF THE INVENTION

The present disclosure provides a modified NEMO protein, constructs encoding the same, and their use to screen and validate inhibitors of the interaction between NEMO and IKK and for structure determination of NEMO in the apo-form and in complex with a ligand.

### BACKGROUND OF THE INVENTION

The nuclear factor κ B (NF-κB) transcription factor is key to the regulation of multiple cellular processes, including cell proliferation and survival, B-cell and T-cell maturation, and inflammatory response. In the canonical NF-κB pathway, NF-κB dimers are sequestered in the cytoplasm by the inhibitor of κB molecules (IκB). Activation of the signaling pathway by stimuli including cytokines, pathogens, stress or ultraviolet radiation, is mediated by an essential node, the IκB kinase (IKK) complex, composed of the NF-κB essential modulator (NEMO) and the IKKα and IKKβ kinases. The IKK complex phosphorylates IκB leading to ubiquitination and proteosomal degradation and allowing NF-κB to translocate to the nucleus and activate target genes. Mis-regulated NF-κB activity has been linked to human diseases encompassing inflammatory and autoimmune diseases and cancer, including resistance to conventional cancer therapy, and modulation of the NF-κB pathway has therefore been the focus for possible therapeutic development.

Inhibition of the protein-protein interaction between NEMO and IKKβ represents an attractive therapeutic strategy due to the crucial role of NEMO and its selective involvement in the canonical NF-κB pathway. However, drug discovery efforts have been hampered because NMR and X-ray techniques cannot be utilized in the study of the unbound NEMO protein.

Modified NEMO proteins and constructs encoding the same are needed to enable X-ray structure determination and NMR-based screening.

### SUMMARY OF THE INVENTION

The present disclosure relates to a mutant NF-κB essential modulator (NEMO) protein or fragment thereof.

In certain embodiments, the mutant NEMO protein or fragment thereof comprises a deletion, insertion, and/or substitution of at least one amino acid residue within amino acids 44-111 of wild-type NEMO (SEQ ID NO: 1). In some such embodiments, the mutant NEMO protein or fragment thereof comprises a substitution of at least one amino acid residue within amino acids 44-111 of wild-type NEMO (SEQ ID NO: 1). In some such embodiments, the substitution is at a residue selected from the group consisting of T50, L55, R75, E78, R106, and E110 of wild-type NEMO. Preferably, the mutant NEMO protein or fragment thereof comprises a substitution of at least 2, 3, 4, 5, or 6 amino acid residues at positions T50, L55, R75, E78, R106, and E110 of wild-type NEMO. In certain embodiments, the mutant NEMO protein or fragment thereof further comprises a substitution of at least one non-essential cysteine, such as C76 or C95.

SEQ ID NO: 1 provides amino acids 44-111 of wild-type NEMO and residues T50, L55, R75, E78, R106, and E110 appear in bold, underlined text:

EQGAPETLQRCLEENQELRDAIRQSNQILRERCEELLHFQASQREEKEF
LMCKFQEARKLVERLGLEK

In certain embodiments, the mutant NEMO protein or fragment thereof comprises the amino acid sequence set forth in SEQ ID NO: 2, wherein at least one Xaa at positions 50, 55, 75, 78, 106, 110 of wild-type NEMO is an amino acid that is different from the corresponding amino acid of SEQ ID NO: 1 and, optionally, one or both Cys residues at positions 76 and 95 of wild-type NEMO have been substituted by another amino acid, preferably Ala or Ser.

In certain embodiments, the mutant NEMO protein or fragment thereof comprises an amino acid sequence having at least 80%, at least 85%, at least 90%, or at least 95% to the amino acid sequence set forth in SEQ ID NO: 3. In some such embodiments, the mutant NEMO protein or fragment thereof comprises the amino acid sequence set forth in SEQ ID NO: 3.

In certain embodiments, the mutant NEMO protein or fragment thereof comprises a deletion, insertion, and/or substitution of at least one amino acid residue within amino acids 50-112 of wild-type NEMO (SEQ ID NO: 4). In some such embodiments, the mutant NEMO protein or fragment thereof comprises a substitution of at least one amino acid residue within amino acids 50-112 of wild-type NEMO (SEQ ID NO: 1). In some such embodiments, the substitution is at a residue selected from the group consisting of T50, L55, R75, E78, R106, and E110 of wild-type NEMO. Preferably, the mutant NEMO protein or fragment thereof comprises a substitution of at least 2, 3, 4, 5, or 6 amino acid residues at positions T50, L55, R75, E78, R106, and E110 of wild-type NEMO. In certain embodiments, the mutant NEMO protein or fragment thereof further comprises a substitution of at least one non-essential cysteine, such as C76 or C95.

In certain embodiments, the mutant NEMO protein or fragment thereof comprises the amino acid sequence set forth in SEQ ID NO: 5, wherein at least one Xaa at positions 50, 55, 75, 78, 106, 110 of wild-type NEMO is an amino acid that is different from the corresponding amino acid of SEQ ID NO: 4 and, optionally, one or both Cys residues at positions 76 and 95 of wild-type NEMO have been substituted by another amino acid, preferably Ala or Ser.

In certain embodiments, the mutant NEMO protein or fragment thereof comprises an amino acid sequence having at least 80%, at least 85%, at least 90%, or at least 95% identity to the amino acid sequence set forth in SEQ ID NO: 6. In some such embodiments, the mutant NEMO protein or fragment thereof comprises the amino acid sequence set forth in SEQ ID NO: 6.

In certain embodiments, the mutant NEMO protein or fragment thereof additionally comprises one or more C-terminal or N-terminal amino acids. For example, in some such embodiments, the mutant NEMO protein or fragment thereof additionally comprises an adaptor at the N- and/or C-terminus.

In certain embodiments, the mutant NEMO protein or fragment thereof comprises an amino acid sequence having at least 80%, at least 85%, at least 90%, or at least 95% identity to the amino acid sequence set forth in SEQ ID NO: 7 or 8. In some such embodiments, the mutant NEMO protein or fragment thereof comprises the amino acid sequence set forth in SEQ ID NO: 7. In some such embodiments, the mutant NEMO protein or fragment thereof comprises the amino acid sequence set forth in SEQ ID NO: 8. In certain embodiments, the mutant NEMO protein or fragment thereof comprises the amino acid sequence set forth in SEQ ID NO: 7 or 8, wherein at least one (e.g., one, two, or three) of the residues at the C-terminal end are deleted and/or replaced by one or more amino acid residues. In certain embodiments, the mutant NEMO protein or fragment thereof comprises the amino acid sequence set forth in SEQ ID NO: 7 or 8, wherein at least one (e.g., one, two, or three) of the residues at positions 101-107 (i.e., RKLVEEL of SEQ ID NO: 8) is replaced by a different amino acid residues.

The present disclosure also relates to the use of such mutant NEMO proteins and fragments thereof to screen for compounds that bind to NEMO and/or inhibit the interaction between NEMO and IKKα and/or IKKβ. In certain embodiments, a mutant NEMO protein or fragment thereof is contacted with a candidate compound.

Thus, in one aspect, this disclosure provides a method for screening for a compound that binds to NEMO and/or inhibits the interaction between NEMO and IKKα and/or IKKβ. In certain embodiments, the method comprises contacting a candidate compound with a mutant NEMO protein or fragment disclosed herein. In some such embodiments, the method comprises structure determination by X-ray crystallography. In some such embodiments, the method comprises NMR-based screening and/or NMR-based structure determination.

The present disclosure relates to nucleic acid constructs encoding a mutant NEMO protein or fragment thereof and cells containing such constructs and/or expressing the mutant NEMO protein or fragment thereof.

The modified proteins, constructs, and screening methods for identifying compounds are further described herein.

These and other objects of the invention are described in the following paragraphs. These objects should not be deemed to narrow the scope of the invention.

### BRIEF DESCRIPTION OF THE DRAWINGS

FIG. 1 is a plot showing HSQC (Heteronuclear Single Quantum Coherence) spectra for WT-NEMO(44-111) and ¹⁵N-MP12.

### DESCRIPTION OF THE INVENTION

This detailed description is intended only to acquaint others skilled in the art with the present invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

NEMO is a 419 amino acid protein containing two coiled-coil domains, a leucine zipper domain, and a zinc finger domain in an elongated dimeric structure. The minimal binding domain necessary to recognize IKKβ was identified as residues 44-111 and the structure was reported in complex with the NEMO-binding domain of IKKβ (residues 701-745). The structure displays a four helical bundle in which the two helices of the NEMO (44-111) dimer are intercalated by the two helices of IKKβ with an extensive interaction interface.

A coiled-coil stabilized NEMO construct encompassing the IKKβ-binding region fused to two ideal dimeric coiled-coil adaptors, at the N- and C-terminus, has been described in Guo et al., Biochemistry 53, 6776-6785 (2014), the entire contents of which is herein incorporated by reference. The coiled-coil stabilized NEMO construct was modified by four additional point mutations, designed to improve solution behavior and crystal packing, as described in Barczewski et al., Scientific Reports 9, 2950 (2019), the entire contents of which is herein incorporated by reference.

### A. Definitions

As used in the specification and the appended claims, unless specified to the contrary, the following terms have the meaning indicated:

The term “about” as used herein means approximately, and in most cases within 10% of the stated value.

The terms “crystal” and “crystallized” refer to a protein, such as a mutant NEMO protein, that exists in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins), or molecular assemblies. These three-dimensional arrays are arranged according to specific mathematical relationships that are well-understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. See Giege, R. and Ducruix, A. Barrett, Crystallization of Nucleic Acids and Proteins, a Practical Approach, 2nd ea., pp. 20, 1-16, Oxford University Press, New York, New York, (1999).

The term “detectable label” as used herein refers to a marker coupled to or incorporated into the protein and used for detection or imaging. Examples of such labels include: radiolabel, a fluorophore, a chromophore, or an affinity tag. In some embodiments, the label is a radiolabel used for medical imaging, for example technetium-99 (⁹⁹^{ₘ}Tc) or iodine-123 (I¹²³), or a spin label for nuclear magnetic resonance (NMR) imaging, such as I¹²³, iodine-131 (I¹³¹), indium-111 (In¹¹¹), fluorine-19 (F¹⁹), carbon-13 (C¹³), nitrogen-15 (N¹⁵), oxygen-17 (O¹⁷), gadolinium, manganese, iron, etc.

The term “mutated” or “mutant” in reference to a nucleic acid or a polypeptide refers to the substitution, deletion, or insertion of one or more nucleotides or amino acids, respectively, compared to the naturally occurring (wild-type) nucleic acid or polypeptide. A mutant NEMO protein or fragment thereof includes at least one substitution, deletion, or insertion relative to the corresponding wild-type NEMO protein and, in particular, residues 44-111 or 50-112 of the wild-type human NEMO protein (UniProt Accession No. Q9Y6K9).

The term “operably linked” as used herein refers to the association of polynucleotide sequences so that the function of one is affected by the other. For example, a promoter is operably linked with a coding sequence when it affects the expression of that coding sequence (i.e., that the coding sequence is under the transcriptional control of the promoter). Coding sequences can be operably linked to regulatory sequences in sense or antisense orientation. The polynucleotide molecules may be part of a single contiguous polynucleotide molecule and may be adjacent. For example, a promoter is operably linked to a coding sequence if the promoter modulates transcription of the coding sequence of interest in a cell.

As used herein, the terms “polypeptide”, “peptide”, and “protein” are used interchangeably and include reference to a polymer of amino acid residues. The terms apply to amino acid polymers in which one or more amino acid residue is an artificial chemical analog of a corresponding naturally occurring amino acid, as well as to naturally occurring amino acid polymers. The terms also apply to polymers containing conservative amino acid substitutions such that the protein remains functional.

The term “residue” refers to an amino acid that is incorporated into a protein, polypeptide, or peptide as used herein. The amino acid residue can be a naturally occurring amino acid and, unless otherwise limited, can encompass known analogs of natural amino acids that can function in a similar manner as naturally occurring amino acids.

### B. Modified Proteins

In one aspect, the present disclosure provides a mutant NEMO protein or fragment thereof. In certain embodiments, the mutant NEMO protein or fragment thereof differs from SEQ ID NO: 1 (amino acids 44-111 of wild-type human NEMO) and/or SEQ ID NO: 4 (amino acids 50-112 of wild-type human NEMO) by up to ten amino acid substitutions, preferably by from one to eight amino acid substitutions. In certain embodiments, the mutant NEMO protein or fragment thereof includes substitutions of T50, L55, R75, E78, R106, and/or E110 of wild-type NEMO. Preferably, the mutant NEMO protein or fragment thereof comprises a substitution of at least 2, 3, 4, 5, or 6 amino acid residues at positions T50, L55, R75, E78, R106, and E110 of wild-type NEMO. In certain embodiments, the mutant NEMO protein or fragment thereof further comprises a substitution of at least one non-essential cysteine, such as C76 or C95. In certain embodiments, the mutant NEMO protein or fragment thereof comprises C54 (i.e., Cys 54 is preserved from wild-type NEMO).

Illustrative examples of alterations of the amino acid sequence are insertions or deletions as well as amino acid substitutions. Such substitutions may be conservative, i.e. an amino acid residue is replaced with an amino acid residue having similar chemical or physical properties, such as polarity, charge, and/or size. In certain embodiments, a conservative substitution(s) is one in which an amino acid residue from one of the following four groups is replaced with an amino acid residue from the same group: Group 1: polar, negatively charged residues and their amides (D, N, E, Q); Group 2: polar, positively charged residues (H, R, K); Group 3: hydrophobic residues (I, L, V, C, A, G, M, F, Y, W, H, K, T); and Group 4: non-polar residues (I, L, V, C, A, G, M, F). Further examples of conservative substitutions are the replacements among the members of the following groups: 1) alanine, serine, and threonine; 2) aspartic acid and glutamic acid; 3) asparagine and glutamine; 4) arginine and lysine; 5) isoleucine, leucine, methionine, and valine; and 6) phenylalanine, tyrosine, and tryptophan.

In other embodiments, the amino acid substitution(s) are non-conservative substitutions, i.e., substitutions of one amino acid residue with another amino acid residue having different chemical or physical properties (polarity, charge, and/or size). In some such embodiments, the substitution improves the biochemical, biophysical and/or biological properties of the peptide.

In certain embodiments, a polar, negatively charged residue such as D or E is replaced by a polar, positively charged residue such as H, R, or K. In certain embodiments, a polar, positively charged residue such as H, R, or K is replaced by a polar, negatively charged residue such as D or E.

In certain embodiments, a polar, uncharged residue such as Q, W, Y, T, or N is replaced by a polar, charged residue such as H, R, K, D, or E. In certain embodiments, a polar, charged residue such as H, R, K, D, or E is replaced by a polar, uncharged residue such as Q, W, Y, T, or N.

In certain embodiments, a non-polar, hydrophobic residue such as I, L, V, C, A, G, M, or F is replaced by a polar, charged residue such as H, R, K, D, or E. In certain embodiments, a polar, charged residue such as H, R, K, D, or E is replaced by a non-polar, hydrophobic residue such as I, L, V, C, A, G, M, or F.

In certain embodiments, the mutant NEMO protein or fragment thereof comprises a deletion or substitution of residues 44-49 of wild-type NEMO. In some such embodiments, residues 44-49 of wild-type NEMO are replaced by VARLKK (SEQ ID NO: 10). The VARLKK sequence is a segment of an optimized ideal dimeric coiled coil. In certain embodiments, the VARLKK sequence is in register with the coiled-coil heptad of the mutant NEMO sequence to which it is attached.

In certain embodiments, the mutant NEMO protein or fragment thereof comprises the amino acid sequence set forth in SEQ ID NO: 2, wherein at least one Xaa at positions 50, 55, 75, 78, 106, 110 of wild-type NEMO is an amino acid that is different from the corresponding amino acid of SEQ ID NO: 1 and, optionally, one or both Cys residues at positions 76 and 95 of wild-type NEMO have been substituted by another amino acid, preferably Ala or Ser. In some such embodiments, at least 2, 3, 4, 5, or 6 Xaa at positions 50, 55, 75, 78, 106, 110 of wild-type NEMO is an amino acid that is different from the corresponding amino acid of SEQ ID NO: 1.

In certain embodiments, the mutant NEMO protein or fragment thereof comprises the amino acid sequence set forth in SEQ ID NO: 5, wherein at least one Xaa at positions 50, 55, 75, 78, 106, 110 of wild-type NEMO is an amino acid that is different from the corresponding amino acid of SEQ ID NO: 4 and, optionally, one or both Cys residues at positions 76 and 95 of wild-type NEMO have been substituted by another amino acid, preferably Ala or Ser. In some such embodiments, at least 2, 3, 4, 5, or 6 Xaa at positions 50, 55, 75, 78, 106, 110 of wild-type NEMO is an amino acid that is different from the corresponding amino acid of SEQ ID NO: 4.

In certain embodiments, the mutant NEMO protein or fragment thereof comprises at least 1, 2, 3, 4, 5, or 6 amino acid substitutions selected from the group consisting of Thr 50→Glu; Leu 55→Arg; Arg 75→Val; Glu 78→Arg; Arg 106→Glu; and Glu 110→Val and may further include at least one amino acid substitution selected from the group consisting of Cys 75→Ala or Ser and Cys 95→Ala or Ser. In certain embodiments, the mutant NEMO protein or fragment thereof comprises the amino acid substitutions: Thr 50→Glu; Leu 55→Arg; Arg 75→Val; Glu 78→Arg; Arg 106→Glu; and Glu 110→Val. In some such embodiments, the mutant NEMO protein or fragment thereof further comprises the amino acid substitutions: Cys 75→Ser and Cys 95→Ala.

In certain embodiments, the mutant NEMO protein or fragment thereof comprises an amino acid sequence having at least 80%, at least 85%, at least 90%, or at least 95% identity to the amino acid sequence set forth in SEQ ID NO: 7. In some such embodiments, the mutant NEMO protein or fragment thereof comprises at least 1, 2, 3, 4, 5, or 6 substitutions selected from the group consisting of T50E, L55R, R75V, E78R, R106E, and E110V relative to wild-type human NEMO. In some such embodiments, the mutant NEMO protein or fragment thereof comprises a substitution of at least one non-essential cysteine (e.g., C76 or C95) relative to wild-type human NEMO. In some such embodiments, the mutant NEMO protein or fragment thereof comprises the following substitutions relative to wild-type human NEMO: T50E, L55R, R75V, C76S, E78R, C95A, R106E, and E110V. In some such embodiments, the mutant NEMO protein or fragment thereof comprises the amino acid sequence set forth in SEQ ID NO: 7.

In certain embodiments, the mutant NEMO protein or fragment thereof comprises an amino acid sequence having at least 80%, at least 85%, at least 90%, or at least 95% identity to the amino acid sequence set forth in SEQ ID NO: 8. In some such embodiments, the mutant NEMO protein or fragment thereof comprises at least 1, 2, 3, 4, 5, or 6 substitutions selected from the group consisting of T50E, L55R, R75V, E78R, R106E, and E110V relative to wild-type human NEMO. In some such embodiments, the mutant NEMO protein or fragment thereof comprises a substitution of at least one non-essential cysteine (e.g., C76 or C95) relative to wild-type human NEMO. In some such embodiments, the mutant NEMO protein or fragment thereof comprises the following substitutions relative to wild-type human NEMO: T50E, L55R, R75V, C76S, E78R, C95A, R106E, and E110V. In some such embodiments, the mutant NEMO protein or fragment thereof comprises the amino acid sequence set forth in SEQ ID NO: 8.

In certain embodiments, the mutant NEMO protein or fragment thereof additionally comprises one or more C-terminal or N-terminal amino acids. For example, in some such embodiments, the mutant

NEMO protein or fragment thereof additionally comprises at least one amino acid residue at the C- and/or N-terminus that can be useful in detection methods. For example, a tryptophan residue may be included at the N-terminus of the mutant NEMO protein or fragment thereof for UV detection. As another example, in some such embodiments, the mutant NEMO protein or fragment thereof additionally comprises an adaptor at the N- and/or C-terminus.

In certain embodiments, the mutant NEMO protein or fragment thereof comprises an adaptor, such as a coiled-coil adaptor, at the C- and/or N-terminus. For example, the mutant NEMO protein or fragment thereof may incorporate an ideal coiled-coil sequence based on GCN4 at the N-terminus, C-terminus, or both termini. Exemplary coiled-coil adaptors include SEQ ID NOs: 9-12. In certain embodiments, the mutant NEMO protein or fragment thereof comprises an optimized coiled coil sequence at the N-terminus, C-terminus, or both termini. In some such embodiments, the optimized coiled-coil sequence is in register with the coiled-coil heptad of the NEMO sequence.

A further aspect of this disclosure provides a nucleic acid (for example a polynucleotide) encoding a mutant NEMO protein or fragment thereof. The polynucleotide may be, for example, DNA, cDNA, PNA, RNA or combinations thereof, either single- and/or double-stranded, or native or stabilized forms of polynucleotides, such as, for example, polynucleotides with a phosphorothioate backbone and it may or may not contain introns so long as it codes for the peptide.

In certain embodiments, a mutant NEMO protein or fragment thereof is encodable by a polynucleotide. Generally, DNA encoding the mutant NEMO protein or fragment thereof may be used in accordance with known techniques, appropriately modified in view of the teachings contained herein, to construct an expression vector, which is then used to transform an appropriate host cell for the expression and production of the mutant NEMO protein or fragment thereof. Thus, a still further aspect of this disclosure provides an expression vector capable of expressing a mutant NEMO protein or fragment thereof.

Generally, DNA encoding the mutant NEMO protein or fragment thereof can be inserted into an expression vector, such as a plasmid, in proper orientation and correct reading frame for expression. The coding sequence may be operably linked to an appropriate transcriptional and translational regulatory control nucleotide sequence, such as a promoter, enhancer, intron, and/or poly A signal. The vector can be introduced into the host through standard techniques. Generally, it may be desirable to select for transformed host cells. One selection technique involves incorporating into the expression vector a DNA sequence, with appropriate regulatory elements, that codes for a selectable trait in the transformed cell, such as antibiotic resistance.

In certain embodiments, the construct comprises a cleavage site, such as a tobacco etch virus (TEV) protease cleavage site. As a result, the mutant NEMO protein or fragment thereof may comprise, for example, at least one amino acid residue before the NEMO sequence as a result of enzymatic cleavage.

Host cells that have been transformed by recombinant DNA can be cultured for a sufficient time and under appropriate conditions known to those skilled in the art in view of the teachings disclosed herein to permit the expression of the mutant NEMO protein or fragment thereof, which can then be recovered.

Alternatively, a mutant NEMO protein or fragment thereof may be synthesized using solid-phase synthesis such as the Fmoc mode of solid-phase peptide synthesis. Temporary N-amino group protection is afforded by the 9-fluorenylmethyloxycarbonyl (Fmoc) group.

In at least one aspect, the present disclosure provides a crystal structure of a mutant NEMO protein or fragment thereof that has a space group of ‘P 1’ (number 1), with unit cell dimensions of a=37.51 Å, b=40.89 Å, c=50.15 Å, α=92.62°, β=106.14°, δ=98.87°. In some such embodiments, the crystal diffracted x-rays to a resolution of about 1.436 Å.

In certain embodiments, the crystallized mutant NEMO protein or fragment thereof comprises amino acids 44-111 of wild-type human NEMO (SEQ ID NO: 1) and has up to ten amino acid substitutions relative to wild-type human NEMO, preferably by from one to eight amino acid substitutions. In some such embodiments, the crystallized mutant NEMO protein or fragment thereof includes substitutions of T50, L55, R75, E78, R106, and/or E110 of wild-type NEMO.

Various methods of protein crystallization are known. Giege et al. (1994) Acta Crystallogr. D50:339; McPherson (1990) Eur. J. Biochem. 189:1. Such techniques include hanging drop vapor diffusion (McPherson (1976) J. Biol. Chem. 251:6300), sitting drop vapor diffusion, microbatch and dialysis.

In certain embodiments, the mutant NEMO protein or fragment thereof, is capable of forming a crystal. Such crystal may be obtained by producing and/or purifying the mutant NEMO protein or fragment thereof and then subjecting the purified mutant NEMO protein or fragment thereof to conditions which promote cyrstallization, thereby obtaining said cyrstal.

It will be readily apparent to those skilled in the are that the unit cells of the crystal compositions may deviate ±1-2 Å or ±1-2° from the above cell dimensions depending on the deviation in the unit cell calculations.

### C. Methods Of Use

In at least one aspect, the present disclosure includes a method for screening for a compound that binds to NEMO and/or inhibits the interaction between NEMO and IKKα and/or IKKβ. In certain embodiments, the method comprises contacting a mutant NEMO protein or fragment thereof with a candidate compound.

In certain embodiments, the method comprises measuring the ability of a candidate compound to bind to the mutant NEMO protein or fragment thereof. In certain embodiments, the method comprises measuring the ability of a candidate compound to block or destabilize the interaction between NEMO and IKKα and/or IKKβ.

In certain embodiments, the mutant NEMO protein or fragment thereof as described herein comprises a detectable label. In some such embodiments, the detectable label may be, for example, a radioactive isotopes such as ²¹¹At, ¹³¹I, ⁹⁰Y, ¹⁸⁶Re, ¹⁸⁸Re, ¹⁵³Sm, ²¹²Bi, ³²P, ²¹²Pb and radioactive isotopes of Lu. In some such embodiments, the radioactive atom is for scintigraphic studies, for example ¹²³I, or a spin label for nuclear magnetic resonance (NMR), such as ¹²³I, ¹³¹I, ¹⁹F, ¹³C, ¹⁵N, or ¹⁷⁰O.

In certain embodiments, the mutant NEMO protein or fragment thereof comprises ¹³C, ²H, or ¹⁵N as a detectable label. In some such embodiments, the mutant NEMO protein or fragment thereof comprises ¹⁵N as a detectable label. A mutant NEMO protein or fragment thereof comprising ¹⁵N can be expressed and purified in a similar manner to the unlabeled version, except, for example, utilizing medium supplemented with ¹⁵NH₄Cl (CIL) for cell growth. In certain embodiments, the mutant NEMO protein or fragment thereof comprising ¹³C, ²H, or ¹⁵N as a detectable label is for use in an NMR study.

In certain embodiments, the screening method comprises determining binding between the candidate compound and the mutant NEMO protein or fragment thereof. In some such embodiments, binding is detected by an NMR method.

NMR spectroscopy is a powerful tool for drug discovery, including fragment-based drug discovery. While NMR has been traditionally used to elucidate the three-dimensional structures and dynamics of biomacromolecules and their interactions, it can also be a very valuable tool for the reliable identification of small molecules that bind to proteins and for hit-to-lead optimization.

In certain embodiments, the screening method comprises structural characterization of the complex formed by the candidate compound and the mutant NEMO protein or fragment thereof, such as by NMR or X-ray crystallography. Complex structure determination by NMR or X-ray crystallography offers the possibility to rationalize the binding interactions and improve binding in a new cycle of design and synthesis.

In certain embodiments, the screening method comprises NMR analysis that uses transverse relaxation-optimized spectroscopy (TROSY).

In certain embodiments, the screening method comprises NMR-based screening using ligand-detected methods (STD-NMR, WaterLOGSY) and/or protein-detected methods (HSQC/TROSY) using a mutant NEMO protein or fragment thereof disclosed herein, which provide high quality NMR spectra.

The mutant NEMO protein or fragment thereof disclosed herein allows structure determination of the IKK-binding domain of NEMO in the free and bound state and give NMR spectra of quality amenable for protein-detected NMR screening (HSQC/TROSY).

In certain embodiments, the ability of a candidate compound to inhibit the NEMO-IKKβ interaction is verified by a competition assay.

In at least one aspect, the present disclosure includes a method for identifying a compound that binds to NEMO and/or inhibits the interaction between NEMO and IKKα and/or IKKβ. In certain embodiments, the method comprises contacting a mutant NEMO protein or fragment thereof with a candidate compound.

In at least one aspect, the present disclosure includes a method for screening, identifying, designing, or optimizing a compound that binds to NEMO and/or inhibits the interaction between NEMO and IKKα and/or IKKβ. In certain embodiments, the method comprises contacting a mutant NEMO protein or fragment thereof with a candidate compound.

In at least one aspect, the structure information or atomic spatial relationship data disclosed herein can be used (e.g., in conjunction with a computer) for screening, identifying, designing, and/or optimizing a compound that binds to NEMO and/or inhibits the interaction between NEMO and IKKα and/or IKKβ. For example, the method for screening, identifying, designing, or optimizing may comprise computationally screeing at least one candidate compound, and preferably a plurality of candidate compounds, using a three-dimensional structural representation of the mutant NEMO protein or fragment thereof. The method may further comprise designing and/or optimizing one more candidate compounds for binding to the mutant NEMO protein or fragment thereof. Thus, the present disclosure includes a method for evaluating the potential of a candidate compound to associate with a mutant NEMO protein or fragment thereof.

### D. Kits

In at least one aspect, the present disclosure provides a kit for screening for a compound that binds to NEMO and/or inhibits the interaction between NEMO and IKKα and/or IKKβ. In certain embodiments, the kit comprises a container containing a mutant NEMO protein or fragment thereof as described herein. In some such embodiments, the mutant NEMO protein or fragment thereof is provided in solution. In some such embodiments, the mutant NEMO protein or fragment thereof is provided in lyophilized form. Optionally, the kit comprises a second container containing a diluent or reconstituting solution for the lyophilized formulation. In certain embodiments, the kit comprises instructions for use of the solution or reconstitution and/or use of the lyophilized formulation. Suitable containers include, but are not limited to, bottles, vials, syringes, and test tubes. The container may be formed from a variety of materials such as glass or plastic.

| SEQ ID | Description |
|---|---|
| 1 | wild type NEMO(44-111) |
| 2 | mutant NEMO(44-111) with Xaa at positions 50, 55, 75, 76, 78, 95, 106, 110 |
| 3 | mutant NEMO(44-111) with T50E, L55R, R75V, C76S, E78R, C95A, R106E, & E110V |
| 4 | wild type NEMO(50-111) |
| 5 | mutant NEMO(50-111) with Xaa at positions 50, 55, 75, 76, 78, 95, 106, 110 |
| 6 | mutant NEMO(50-111) with T50E, L55R, R75V, C76S, E78R, C95A, R106E, & E110V |
| 7 | MP11 |
| 8 | MP12 |
| 9 | VARLKK |
| 10 | GSWVARLKK |
| 11 | coil-coiled adaptor (C term) |
| 12 | coil-coiled adaptor (N term) |

It will be readily apparent to those skilled in the art that other suitable modifications and adaptations of the compositions and methods of the invention described herein may be made using suitable equivalents without departing from the scope of the invention or the embodiments disclosed herein.

The compositions and methods described herein will be better understood by reference to the following examples, which are included as an illustration of and not a limitation upon the scope of the invention.

### E. Examples

### Example 1

The natural sequence of the protein NEMO/IKKγ was modified. The modifications include point mutations, insertions, deletions and addition of adaptors. The modifications resulted in mutant NEMO proteins or fragments having 1) modified secondary and tertiary structure: improved helical content, coiled-coil structure and dimerization propensity; 2) improved stability; 3) improved solubility and reduced tendency to aggregate; 4) reduced conformational exchange; 5) capability to generate high quality NMR spectra; 6) capability to enable X-ray crystallography structure determination; and 7) improved affinity for IKKβ and fragments thereof. The modifications represent a novel approach to engineering NEMO to enable biochemical and biophysical studies and further investigate its role in the NF-κB pathway.

The amino acid sequence of exemplary proteins (MP11, SEQ ID NO: 7 and MP12, SEQ ID NO: 8) are shown relative to the amino acid sequence of wild-type human NEMO(44-111) (SEQ ID NO: 1):

```
    44 50 60 70 80 90 100 110
      EQGAPET LQRCLEENQE LRDAIRQSNQ ILRERCEELL HFQASQREEK EFLMCKFQEA RKLVERLGLE K (1)
GSWVARLKKE LQRCREENQE LRDAIRQSNQ ILREVSERLL HFQASQREEK EFLMAKFQEA RKLVEELGLV KLE (7)
GSW------E LQRCREENQE LRDAIRQSNQ ILREVSERLL HFQASQREEK EFLMAKFQEA RKLVEELGLV KLE (8)
```

Constructs were cloned into a vector with a cleavage site inserted before the NEMO sequence. A tryptophan was inserted at the N-terminus of the NEMO construct for UV detection. The residues GSW before the NEMO sequence was a result of the protease cleavage and W insertion. While the W was introduced for UV detection, it also stabilized the structure and participated in crystal contacts between dimers.

The resulting constructs were expressed, the mutant proteins purified and tested against the 7 features listed above, and were shown to be successful.

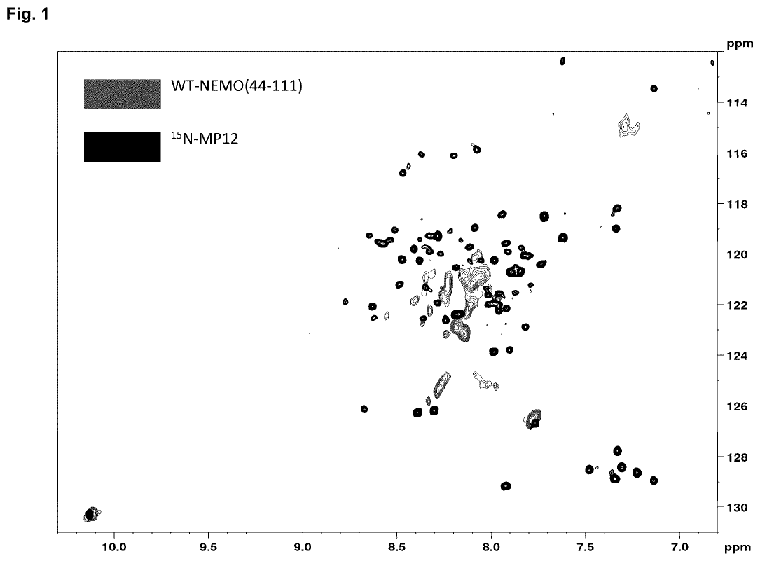

FIG. 1 is a plot that demonstates improved screening of potential small moelcule leads by MP12 (SEQ ID NO: 8) relative to wild-type human NEMO(44-111) (SEQ ID NO: 1).

In red: 1H, 15N HSQC spectrum of WT-NEMO(44-111) shows only diffuse density, typical of a molecule displaying conformational heterogeneity and line broadening due to conformational exchange. Such spectrum cannot be used for 1H, 15N NMR-based screening or lead validation.

The black spectrum of MP12 shows the expected number of cross peak for the protein sequence with sharp and well resolved peaks. This spectrum can be used for NMR-based screening and mapping of the ligand binding site once resonance assignment is completed.

The mutant NEMO proteins and fragments thereof that are disclosed herein enable biochemical assays, NMR studies and X-ray crystallography in manners that could not be achieved with wild-type NEMO. Moreover, the mutant NEMO proteins and fragments thereof that are disclosed herein enable screening and validation of NEMO inhibitors and structure determination of NEMO in the apo-form and in complex with ligands/inhibitors. Such proteins and fragments are also easy to express and purify and are stable at high concentrations.

Thus, mutant NEMO proteins and fragments thereof that are disclosed herein can be utilized for biochemical characterization, cellular characterization, NMR-based screening, NMR-based and X-ray crystallography structure determination in the apo form and in complex with natural partners, peptide ligands and inhibitors.

### Example 2

The crystal structure of MP12 was determined.

The MP12 sample was labeled with SeMet. Crystals were grown by sitting drop vapor diffusion in the following conditions:
40% Morpheus precipitant mix 7
0.1 M Morpheus buffer system 4 pH 6.5
0.00266 M lanthanides
1 M L-Proline

Four datasets acquired on the same crystal at the FMX beamline at NSLSII were processed with Autoproc (Global Phasing Limited) and merged using Blend (J. Foadi, P. Aller, Y. Alguel, A. Cameron, D. Axford, R.L. Owen, W. Armour, D.G. Waterman, S. Iwata and G. Evans Clustering procedures for the optimal selection of data sets from multiple crystals in macromolecular crystallography” Acta Cryst. (2013), D69, 1617-1632). The data was the anisotropically truncated using Staraniso (Global Phasing Limited).
Cell dimensions: a=37.5135 Å, b=40.8918 Å, c=50.1497 Å, α=92.6161°, β=106.1352°, γ=98.8731°.
Space group =‘P 1’ (number 1)

Diffraction limits & principal axes of ellipsoid fitted to diffraction cut-off surface:

| | | | | |
|---|---|---|---|---|
| 1.878 | 0.8852 | 0.1429 | −0.4427 | 0.696 _a_* + 0.004 _b_* − 0.718 _c_* |
| 1.395 | 0.1351 | 0.8316 | 0.5387 | 0.131 _a_* + 0.844 _b_* + 0.521 _c_* |
| 1.671 | 0.4452 | −0.5366 | 0.7168 | 0.392 _a_* − 0.575 _b_* + 0.718 _c_* |

The final data included a resolution range: (47.965-1.436 Å).

The data was phased by SAD using the HySS module of Phenix.

The structure of the unbound MP12 is an open dimer and, thus, can be used for co-crystallization with small molecule and peptide ligands.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations, or methods, or any combination of such changes and modifications of use of the invention, may be made without departing from the spirit and scope thereof.

All references (patent and non-patent) cited above are incorporated by reference into this patent application. The discussion of those references is intended merely to summarize the assertions made by their authors. No admission is made that any reference (or a portion of any reference) is relevant prior art (or prior art at all). Applicant reserves the right to challenge the accuracy and pertinence of the cited references.

1. A mutant NF-κB essential modulator (NEMO) protein or fragment thereof comprising a substitution of at least one amino acid residue within amino acids 44-111 of wild-type NEMO (SEQ ID NO: 1), wherein the at least one amino acid residue is selected from the group consisting of T50, L55, R75, E78, R106, and E110.

2. The mutant NEMO protein or fragment of claim 1, wherein
a. the Thr at position 50 is replaced by a negatively charged amino acid;
b. the Leu at position 55 is replaced by a positively charged amino acid;
c. the Arg at position 75 is replaced by a non-polar or hydrophobic amino acid;
d. the Glu at position 78 is replaced by a positively charged amino acid;
e. the Arg at position 106 is replaced by a negatively charged amino acid; and/or
f. the Glu at position 110 is replaced by a non-polar or hydrophobic amino acid.

3. The mutant NEMO protein or fragment of claim 1, wherein the mutant NEMO protein or fragment thereof comprises at least one substitution selected from the group consisting of T50E, L55R, R75V, E78R, R106E, and E110V.

4. The mutant NEMO protein or fragment of claim 1, wherein the mutant NEMO protein or fragment thereof further comprises a substitution of at least one non-essential cysteine.

5. The mutant NEMO protein or fragment of claim 4, wherein the non-essential cysteine is C76 or C95.

6. The mutant NEMO protein or fragment of claim 1, wherein the mutant NEMO protein or fragment thereof comprises at least one substitution selected from the group consisting of T50E, L55R, R75V, C76S, E78R, C95A, R106E, and E110V.

7. The mutant NEMO protein or fragment of claim 1, wherein the mutant NEMO protein or fragment thereof comprises a deletion or substitution of residues 44-49 of wild-type NEMO.

8. The mutant NEMO protein or fragment of claim 7, wherein residues 44-49 of wild-type NEMO are replaced by VARLKK (SEQ ID NO: 10).

9. The mutant NEMO protein or fragment of claim 1, wherein the mutant NEMO protein or fragment thereof is capable of forming a complex with inhibitor of nuclear factor kappa-B kinase subunit α (IKK1) and inhibitor of nuclear factor kappa-B kinase subunit β (IKK2).

10. The mutant NEMO protein or fragment of claim 1, further comprising an adaptor.

11. A polynucleotide encoding the mutant NEMO protein or fragment of claim 1.

12. A vector or host cell comprising the polynucleotide of claim 11.

13. A method for screening a candidate compound for binding to NEMO and/or inhibiting interaction between NEMO and IKKα and/or IKKβ, the method comprising contacting the candidate compound with the mutant NEMO protein or fragment of claim 1.

14. The method of claim 13, further comprising structure determination by X-ray crystallography.

15. The method of claim 13, further comprising generating NMR-based screening and/or NMR-based structure determination.

16. A crystal comprising a mutant NF-κB essential modulator (NEMO) protein or fragment thereof, wherein said crystal is characterized by a space group of ‘P 1’ (number 1), with unit cell dimensions of a=37.51 Å, b=40.89 Å, c=50.15 Å, α=92.62°, β=106.14°, γ=98.87°.

17. The crystal of claim 16, wherein the mutant NEMO protein or fragment thereof comprises a substitution of at least one amino acid residue within amino acids 44-111 of wild-type NEMO (SEQ ID NO: 1), wherein the at least one amino acid residue is selected from the group consisting of T50, L55, R75, E78, R106, and E110.

18. The crystal of claim 17, wherein
a. the Thr at position 50 is replaced by a negatively charged amino acid;
b. the Leu at position 55 is replaced by a positively charged amino acid;
c. the Arg at position 75 is replaced by a non-polar or hydrophobic amino acid;
d. the Glu at position 78 is replaced by a positively charged amino acid;
e. the Arg at position 106 is replaced by a negatively charged amino acid; and/or
f. the Glu at position 110 is replaced by a non-polar or hydrophobic amino acid.

19. The crystal of claim 16, wherein the mutant NEMO protein or fragment thereof comprises at least one substitution selected from the group consisting of T50E, L55R, R75V, E78R, R106E, and E110V.

20. A method for screening a candidate compound for binding to NEMO and/or inhibiting interaction between NEMO and IKKα and/or IKKβ, the method comprising contacting the candidate compound with the mutant NEMO protein or fragment thereof, wherein the mutant NEMO protein or fragment thereof comprises a substitution of at least one amino acid residue within amino acids 44-111 of wild-type NEMO (SEQ ID NO: 1), wherein the at least one amino acid residue is selected from the group consisting of T50, L55, R75, E78, R106, and E110.
